(19) United States
(12) Patent Application Publication
(10) Pub. No.: US 2024/0308880 A1
(43) Pub. Date: Sep. 19, 2024

(54) METHODS AND DEVICES FOR ULTRAVIOLET FLUID TREATMENT OF FLUIDS

(71) Applicant: TROJAN TECHNOLOGIES GROUP ULC, London (CA)

(72) Inventors: Michael SASGES, London (CA); John VAREKA, London (CA); Ana-Maria BOGATAN, London (CA)

(73) Assignee: TROJAN TECHNOLOGIES GROUP ULC, London, ON (CA)

(21) Appl. No.: 18/578,477

(22) PCT Filed: Jul. 13, 2022
(86) PCT No.: PCT/IB2022/056472

§ 371 Date: Jan. 11, 2024

Related U.S. Application Data
(60) Provisional application No. 63/221,195, filed on Jul. 13, 2021.
(60) Provisional application No. 63/221,221, filed on Jul. 13, 2021.

(51) Int. Cl. C02F 1/32; A61L 2/10; A61L 2/26; C02F 1/00

(52) U.S. Cl. CPC C02F 1/325; A61L 2/10; A61L 2/26; C02F 1/008; A61L 2202/11; A61L 2202/121; A61L 2202/122; A61L 2202/123; A61L 2202/14; C02F 2201/004; C02F 2201/3222; C02F 2201/3228; C02F 2201/326; C02F 2201/328; C02F 2303/04

(57) ABSTRACT

A fluid treatment system for treating a fluid with ultraviolet light. The system includes a detachable light source assembly having a thermally conductive outer wall, an inner wall disposed inside the outer wall and surrounding an inner space within the inner wall that defines a treatment zone, and an ultraviolet light source disposed between the outer wall and the inner wall and configured to emit ultraviolet light toward the treatment zone, and a reactor vessel having an inlet port for receiving a fluid and an outlet port for expelling treated fluid, the reactor vessel configured to receive the light source assembly in an attached state such that a space between a wall of the reactor vessel and the outer wall of the light source assembly defines a cooling zone in which the outer wall is cooled by contacting the fluid.

### CROSS-REFERENCE TO RELATED APPLICATIONS

This application claims priority to Provisional Application No. 63/221,195, filed Jul. 13, 2021, and Provisional Application No. 63/221,221, filed Jul. 13, 2021. The content of the prior applications are hereby incorporated by reference herein in their entirety.

### TECHNICAL FIELD

This application relates systems and methods for treating fluids with ultraviolet light.

### BACKGROUND

The proper disinfection of water is critical to ensure water quality. As the need for cleaner sources of water has increased, water disinfection methods have evolved to match the rising challenge. Water sources may contain heavy metals, sediment, chemicals, pesticides, or the like. Water sources may also contain pathogens such as microorganisms, viruses, or the like. Left untreated, such water may be unhealthy or unsafe for use by humans or animals. Ultraviolet light treatment of water may be used to inactivate pathogens. Water may pass through a small chamber or a larger vessel where the water is subjected to ultraviolet light. The ultraviolet treatment may damage nucleic acids of the pathogens. The disruption of the genetic material may render the pathogens incapable of performing vital cellular functions, thereby rendering them harmless. Thus, this ultraviolet treatment process may make water potable despite the water source containing microorganisms, viruses, or the like.

Ultraviolet treatment may be used in residential, municipal, commercial, industrial, agricultural, medical, food processing facilities, or the like. Ultraviolet treatment may be used for water re-use, soil decontamination, and waste-water treatment applications. Ultraviolet may also be used to treat contaminants in water, such as trace amounts of pesticides, solvents, or other organic molecules. In this case, the ultraviolet wavelengths are used to directly affect chemical bonds of the contaminant, a process called photolysis, or by acting on water itself to create radical species, or by converting an added chemical such as hydrogen peroxide or ozone to a radical species, such as OH radicals, that react with the contaminant to convert it to compounds that are more desirable or less harmful.

Ultraviolet light, e.g., wavelength in the range of 100 to 400 nanometers, may be provided by a variety of ultraviolet light sources such as, for example, ultraviolet light emitting diodes (LEDs), excimer lamps, mercury vapor lamps, or the like. Conventional ultraviolet light source arrangements vary depending on the application. For example, a residential ultraviolet light source may be used to treat well water or other non-municipal water source. Residential units may have a chamber containing an ultraviolet light source piped in-line between a water source, such as a well, and the household piping. A residential system may have one or more ultraviolet lights that expose the water to ultraviolet light as the water passes through the ultraviolet purification chamber. Residential systems may be designed for the relatively lower volume of water used by a home as compared to a larger industrial or commercial ultraviolet treatment system. Larger ultraviolet treatment systems may be found in industrial or commercial facilities such as a municipal water treatment facility. The larger systems may handle large volumes of water, and may, therefore, have a plurality of ultraviolet lamps, for example, as arranged in an array. These ultraviolet light sources may be arranged in or around vessels of water to be treated with ultraviolet light. Commercial systems (see U.S. Pat. Nos. 8,246,839 and 7,077,965) may have multiple vessels or tubes through which water passes for ultraviolet treatment.

Conventional ultraviolet disinfection systems have some drawbacks. Ultraviolet disinfection may be performed using a reactor. A reactor may be a chamber or series of chambers to treat the fluid, or the like, with an ultraviolet source. Conventional reactors include axial flow reactors. Axial flow reactors have a plurality of chambers configured in an axial configuration, or along an axis. Axial reactors represent a loss of space and compactness as the components or chambers may be spread along the length of flow of a fluid. In some applications, space may be at a premium along piping. For example, in a residential setting, a retrofit in which ultraviolet disinfection is added or upgraded, or the like, there may not exist physical space to allow for an axial reactor to be installed. Thus, installation of a conventional reactor may cause problems and require replumbing and/or reconfiguration of a fluid flow.

Additionally, conventional ultraviolet reactors may be difficult to cool. Proper disinfection requires a minimum amount of light output from the ultraviolet source upon the fluid to be treated. The ultraviolet light source produces heat. This heat may cause damage to surfaces of the reactor and surrounding areas. Some systems have employed a cooling system for the ultraviolet source such as those that individually cool ultraviolet light sources. (See U.S. Pat. No. 10,604,423). Systems with an active cooling system for the ultraviolet source add complexity, cost, and maintenance to a system. Also, conventional ultraviolet reactors may have an ultraviolet source along the exterior. For example, an ultraviolet illumination source may be placed on the surface of a transparent material to disinfect a fluid within the material. The ultraviolet light may damage or degrade the transparent material.

Furthermore, conventional systems may contain ultraviolet light sources inside a pipe. Systems may incorporate LED sources which irradiate through a quartz sleeve into a flowing liquid. The liquid pressure may break the quartz sleeve due to the quartz sleeve being poorly suited to resisting internal pressure. For an example of such an arrangement, refer to U.S. Pat. No. 10,604,423. For some applications, the pressure within a pipe may be high. These pressures make it difficult to incorporate a quartz sleeve and an LED layer onto a structural pipe member, especially if the pipe is constructed of a metal. What is needed is an ultraviolet treatment reactor with a cooled ultraviolet source that is pressure balanced.

### SUMMARY

These and other problems are addressed by the disclosed systems and methods for treating a fluid in a fluid system.

In a first embodiment, there is provided a fluid treatment system for treating a fluid with ultraviolet light. The system includes at least one detachable light source assembly having a thermally conductive outer wall, an inner wall disposed inside the thermally conductive outer wall and surrounding an inner space that defines a treatment zone, and an ultraviolet light source disposed between the thermally conductive outer wall and the inner wall and configured to emit ultraviolet light toward a center of the treatment zone in order to treat the fluid, and a reactor vessel having an inlet port for receiving a fluid into the reactor vessel and an outlet port for expelling treated fluid from the reactor vessel, the reactor vessel configured to receive the light source assembly in an attached state such that a space between a wall of the reactor vessel and the thermally conductive outer wall of the light source assembly defines a cooling zone in which the fluid contacts the thermally conductive outer wall before the fluid enters or after the fluid exits the treatment zone to thereby cool the thermally conductive outer wall.

The system may further comprise a plurality of light source assemblies, where the reactor vessel is configured to receive the plurality of light source assemblies in an attached state

The system may further comprise a flow modifying device disposed on a fluid entry side of the light source assembly, the flow modifying device configured to control a flow of the fluid through the treatment zone.

The reactor vessel may include a header portion and a base portion, the header portion and the base portion may each further include a connector portion configured to connect the header portion and the base portion and in the attached state. The connector portion may be selected from the group consisting of a press fit, a thread, a bayonet lock, a threaded ring, and a sanitary clamp. An o-ring may seal the header portion and the base portion in the attached state.

The reactor vessel and the light source assembly may each further include an electrical contact portion configured to electrically connect the light source assembly and the reactor vessel.

The ultraviolet light source may comprise a plurality of light emitting diodes.

The system may further comprise a controller configured to adjust an intensity of the ultraviolet light source based on at least one parameter selected from the group consisting of time, intensity, flow rate, voltage, temperature, ultraviolet intensity, and optical absorbance.

The fluid may turn 180 degrees flowing from the cooling zone to the treatment zone.

The light source assembly may have a cylindrical shape or a rectangular cross-sectional shape.

The thermally conductive outer wall may comprise a material selected from the group consisting of copper, composite, and stainless steel.

An inner surface of the light source assembly may include a reflective material configured to reflect light within the treatment zone.

The reflective material may be at least one selected from the group consisting of fluoropolymer, TiO₂, SiO₂, and BO₂.

The ultraviolet light source may be disposed on an inner surface of the thermally conductive outer wall and is configured to emit the ultraviolet light toward the center of the treatment zone in a radial direction relative to a longitudinal axis of the light source assembly.

The inner wall may comprise a quartz material.

The fluid may be water in a water treatment facility.

In another embodiment, there is provided a method for treating a fluid with ultraviolet light in a fluid treatment system, the system comprising (i) at least one detachable light source assembly having a thermally conductive outer wall, an inner wall disposed inside the thermally conductive outer wall and surrounding an inner space that defines a treatment zone, and an ultraviolet light source disposed between the thermally conductive outer wall and the inner wall, and (ii) a reactor vessel having an inlet port for receiving a fluid into the reactor vessel and an outlet port for expelling treated fluid from the reactor vessel, the reactor vessel configured to receive the light source assembly in an attached state such that a space between a wall of the reactor vessel and the thermally conductive outer wall of the light source assembly defines a cooling zone. The method includes treating the fluid in the treatment zone by emitting ultraviolet light toward the treatment zone, and cooling the fluid in the cooling zone by contacting the fluid with the thermally conductive outer wall before the fluid enters or after the fluid exits the treatment zone to thereby cool the thermally conductive outer wall.

In another embodiment, there is provided a light source assembly for treating a fluid with ultraviolet light. The assembly includes a thermally conductive outer wall, an inner wall disposed inside the thermally conductive outer wall, an inner space surrounded by the inner wall that defines a treatment zone, an ultraviolet light source disposed between the thermally conductive outer wall and the inner wall and configured to emit ultraviolet light toward the treatment zone in order to treat the fluid, and an electrical contact portion configured to detachably electrically connect the light source assembly to an electrical power supply.

The inner wall of the light source assembly may include an ultraviolet-transmissive material selected from the group consisting of quartz, sapphire, and fluoropolymer.

The light source assembly may further include a sensor configured measure an intensity of the ultraviolet light.

The inner wall of the light source assembly may include at least one portion with ultraviolet light reflectivity of at least 50%.

The light source assembly may further include a flow modifying device disposed on a fluid entry side of the light source assembly, the flow modifying device configured to control a flow of the fluid through the treatment zone.

In another embodiment, there is provided a reactor vessel configured to receive at least one detachable light source assembly for treating a fluid with ultraviolet light. The reactor vessel includes an inlet port for receiving a fluid into the reactor vessel, an outlet port for expelling treated fluid from the reactor vessel, a header portion and a base portion, the header portion and the base portion each further including a connector portion configured to connect the header portion and the base portion in an attached state, and an electrical contact portion configured to electrically connect the reactor vessel to the light source assembly. A space between a wall of the reactor vessel and a thermally conductive outer wall of the light source assembly defines a cooling zone in which the thermally conductive outer wall is cooled by contacting the fluid with the thermally conductive outer wall before the fluid enters or after the fluid exits a treatment zone of the light source assembly when the light source assembly is in an attached state.

### BRIEF DESCRIPTION OF THE OF THE DRAWINGS

FIGS. 1A, 1B, and 1C illustrate cutaway views of embodiments of fluid treatment systems having a flow modifier in a first position (FIG. 1A), a flow modifier in a second position (FIG. 1B), and no flow modifier (FIG. 1C).

FIG. 2 illustrates a cutaway view of an embodiment of a fluid treatment system.

FIG. 3 illustrates an exploded view of an embodiment of a fluid treatment system.

FIG. 4 illustrates a top perspective view of an embodiment of a fluid treatment system.

FIG. 5 illustrates electrical connections according to an embodiment.

FIG. 6 illustrates an exploded view of an embodiment of a fluid treatment system.

FIG. 7 illustrates another exploded view of an embodiment of a fluid treatment system.

FIG. 8 illustrates a cutaway view of a light source according to an embodiment of a fluid treatment system.

FIG. 9 illustrates another cutaway view of a light source according to an embodiment of a fluid treatment system.

FIG. 10 illustrates a network according to an embodiment of a fluid treatment system.

FIG. 11 illustrates an embodiment of disclosed methods of fluid treatment.

FIG. 12 illustrates another embodiment of disclosed methods of fluid treatment.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of this disclosure but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

### Introduction

Ultraviolet water treatment systems are a cost effective and efficient method to treat water containing biological contaminants. One treatment includes ultraviolet disinfection. Ultraviolet disinfection may protect against water borne viruses, bacteria, molds, or the like. Additionally, ultraviolet disinfection may be effective against disease-causing organisms such as Giardia and Cryptosporidium. Proper control of ultraviolet light delivered to a volume of water may be essential to the proper treatment of the water. In other words, effective exposure of contaminated water to an ultraviolet source may be critical to proper disinfection of a water source. While the embodiments discussed herein will be described with respect to ultraviolet disinfection, it should be understood that the methods and systems as described herein extend to any ultraviolet treatment and not just disinfection.

This disclosure focuses on the application of ultraviolet treatment of water for the disinfection of drinking water. However, other implementations of the systems and methods described herein are possible and contemplated. For example, since ultraviolet light may be used to disinfect surfaces, the described systems and methods may be used in such an application. These surfaces may include areas used to prepare sensitive materials in which organisms or organic residue would contaminate the surface such as a biological hood, surgical suite, food preparation area, clean room, or the like. Additionally, the described system and method may be used for the disinfection of pool water, the disinfection of a gas (e.g., ultraviolet light may be used to purify air), contaminated soil, in advanced oxidation or environmental contaminant treatment (ECT) applications, ionization of organic molecules, and the like. Thus, the examples described herein regarding the disinfection of water are merely illustrative. Other uses are contemplated and disclosed.

Disclosed systems and methods are directed to a fluid treatment system for the treatment of a fluid using ultraviolet light. The fluid may be water in a water treatment facility. In an embodiment, the fluid treatment system may have a reactor vessel and a light source assembly. The fluid may enter the reactor vessel through an inlet port. From the inlet port, the fluid may enter the cooling zone. The cooling may refer to a cooling of an ultraviolet light source, not the cooling of the fluid itself. For example, the fluid may provide cooling to the ultraviolet light source and the fluid itself may increase in temperature. From the cooling zone, the fluid may enter a treatment zone. A fluid modifier or baffle may be present between the cooling and treatment zones. The fluid may undergo ultraviolet illumination or treatment in the treatment zone. The cooling and treatment zones may be separated by a wall. The wall may have a surface with an ultraviolet source on the treatment zone side, and the wall may provide cooling from fluid in the cooling zone on an opposite side of the wall. From the treatment zone, the fluid may exit the reactor vessel from an outlet port. The fluid may then flow to piping, storage, further treatment/disinfection, or the like.

In this manner, fluid flowing through the cooling chamber may cool the wall on which the ultraviolet source is mounted opposite the wall in the treatment zone. A flow of fluid ensures a continuous supply of cooling fluid to the wall and subsequently to the ultraviolet source. The wall may be of a thermally conductive material. In an embodiment the pressure of a fluid is nearly equal in both the outer cooling and inner treatment chambers. This may be referred to as a pressure balanced reactor.

The reactor vessel and light source assembly may be of any cross-sectional shape. For example, the reactor vessel and light source assembly may be cylindrical, hexagonal, ovoid, right regular prism, or the like. This disclosure may refer to a reactor vessel with a cylindrical cup shape and may have an annular volume, but embodiments should not be so limited.

The reactor vessel and light source assembly may be concentric. In this manner, fluid may travel from an inlet port into the annular space and along the wall that defines the inner diameter of the outer vessel and the outer diameter of the inner vessel. The fluid in the annular space provides a cooling function. As fluid enters the inlet port and outer vessel, the fluid passes by the surface of the cooling zone wall also defining the light source assembly. The surface of the wall facing the inside of the treatment zone may comprise one or more ultraviolet light source(s). In other words, as the ultraviolet light generated in the treatment zone heats the surface of the wall facing the treatment zone, the fluid may cool the wall as the fluid passes the surface of the wall facing the cooling zone.

The fluid may then turn 180 degrees, may pass through a fluid modifier, e.g., a baffle, and enter the light source assembly. The flow modifier or baffle may be an optional element to the device or method. The light source assembly may be a cylindrical tube housed within the reactor vessel. The light source assembly may be of any cross-sectional shape. For example, the light source assembly cross-sectional shape may be a square, hexagonal, right regular prism, or the like. In an embodiment, an LED light source may be mounted upon a flat surface of the cross-sectional shape. In an embodiment, there may be more than one light source assembly within the reactor vessel. The reactor vessel and light source assembly volumes may share a common wall. The wall may have two surfaces. The first surface may partially define the reactor vessel. The second surface may partially define the light source assembly. The wall may have one or more light sources in the wall facing towards the treatment zone. In an embodiment, the light sources may be arranged radially, axially, in a regular pattern such as a grid or checkerboard, or the like. The light sources may be upon the wall comprising thermally conductive material. In this manner, fluid flowing through the reactor vessel or cooling zone may cool the wall and the light source of the treatment zone. The light source may provide a treatment to the fluid in the treatment zone. The fluid may flow out of the treatment zone and through an outlet port.

The reactor vessel may have a header portion that incorporates a receptacle mechanically complementary to a fitting on a proximal end of a light source assembly. The header portion may incorporate a receptacle mechanically complimentary to a fitting on the open end of a reactor vessel. The header portion may also attach to the reactor vessel and/or light source assembly using a thread, bayonet lock, threaded ring, sanitary clamp, press fit, snap fit, or the like. The header portion may also include electrical connections or electrical pins of the light source assembly protruding from the reactor vessel, as will be described herein. Attachment points may have associated O-rings, gaskets, or the like. There may be an attachment for different regions of the reactor vessel and/or light source assembly. Attachment and/or detachment of the reactor vessel from light source assembly may not require full rotation of either component such that the component does not require a full turn (360 degree) or spin. In an embodiment, attachment of the reactor vessel may hold the light source assembly in place.

In an embodiment, the header portion may serve multiple functions. The header portion may contain the inlet port and/or the outlet port. The header region may comprise electrical connections for the ultraviolet light source, other fluid characteristic sensors, or the like. For example, electrical connections in the form of pins and complementary contacts. The electrical connection may be pins, wiping contacts, tabs, plugs, or the like to provide electrical continuity between reactor components. In an embodiment, the electrical connection may provide connection from an electrical driver, such as a current source or power source, to the ultraviolet light source such as ultraviolet LED.

The illustrated example embodiments will be best understood by reference to the figures, as described below. The following description exemplifies certain embodiments but it will be understood that this disclosure is not intended to be limited to these specific embodiments.

### The Fluid Treatment System

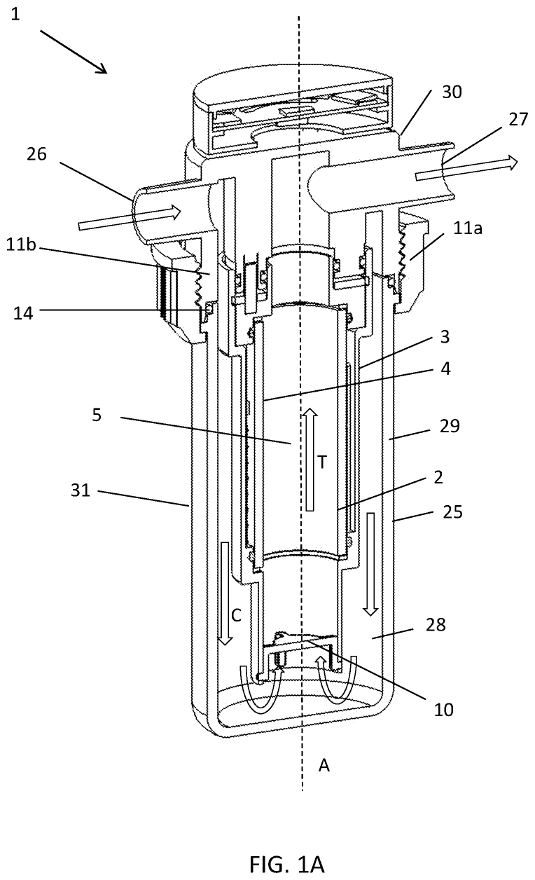

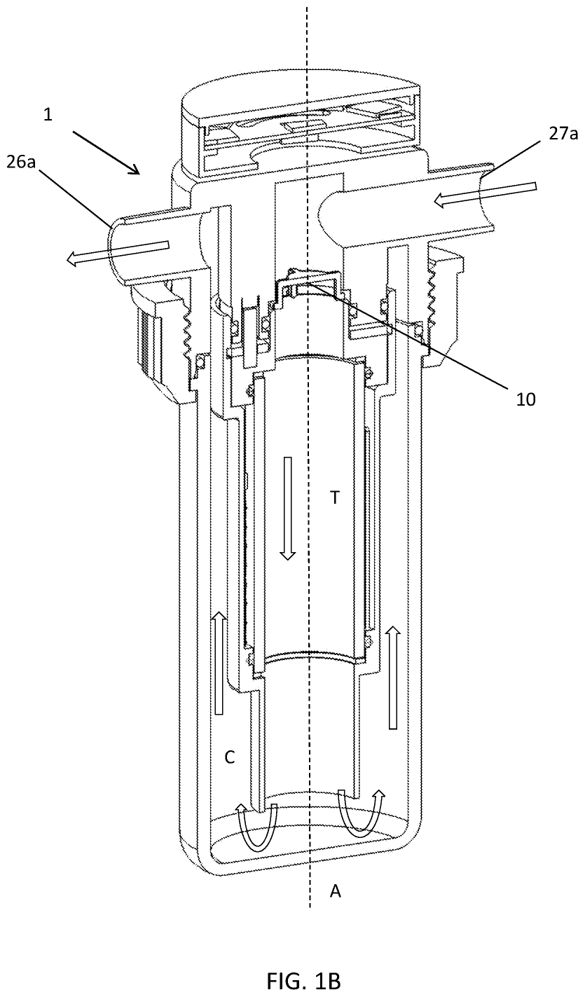

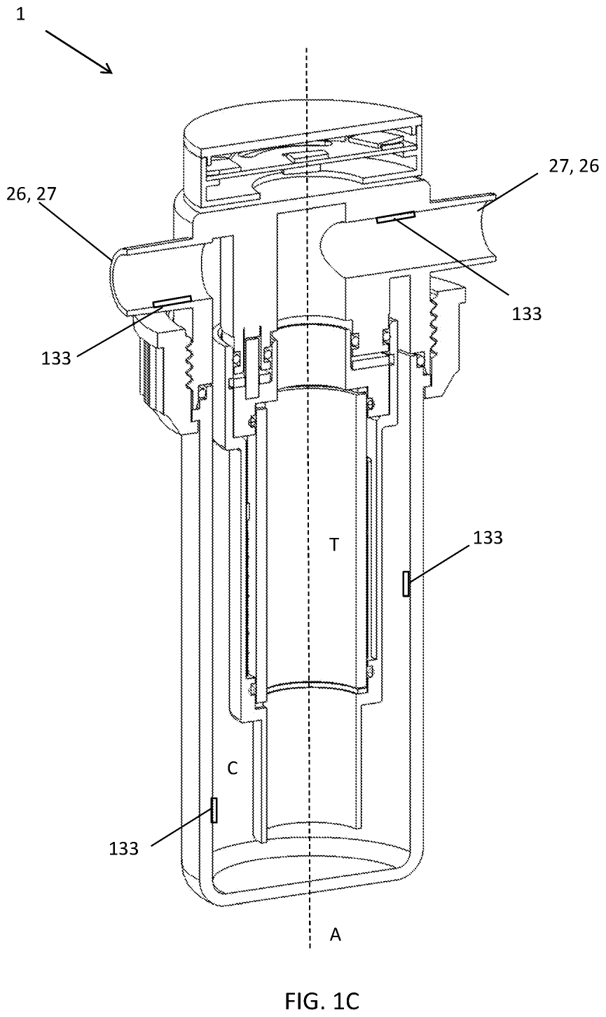

Disclosed embodiments include a fluid treatment system 1 for treating a fluid with ultraviolet light. As seen in FIGS. 1A, 1B, and 1C, the fluid treatment system 1 may include at least one detachable light source assembly 2 and a reactor vessel 25, both of which are further described in detail below. In this embodiment, the reactor vessel 25 is configured to receive the light source assembly 2 in an attached state such that a space 28 between a wall 29 of the reactor vessel 25 and a thermally conductive outer wall 3 of the light source assembly 2 defines a cooling zone C in which the thermally conductive outer wall 3 is cooled by contacting the thermally conductive outer wall 3 with the fluid before the fluid enters or after the fluid exits a treatment zone T.

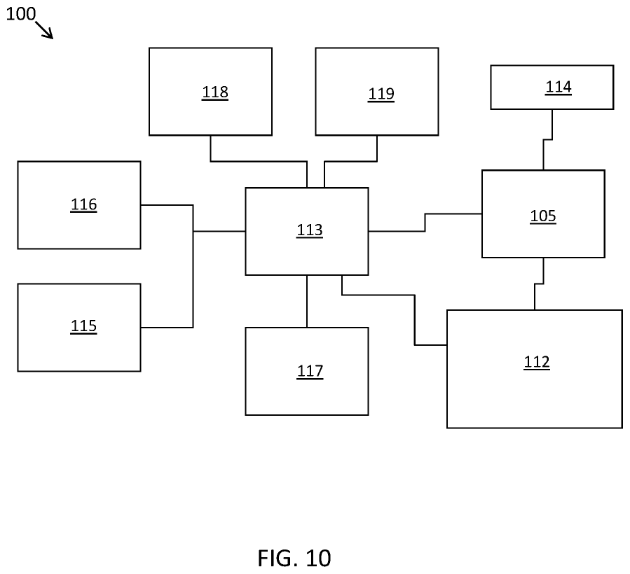

The fluid treatment system 1 may further include a network 100 including a controller 113 (described further below with reference to FIG. 10) configured to control functioning of the fluid treatment system 1 including adjusting an intensity of an ultraviolet light source 6 of the light source assembly 2. Control in this respect may be based on at least one parameter selected from the group consisting of time, intensity, flow rate, voltage, temperature, ultraviolet intensity, and optical absorbance.

### The Light Source Assembly

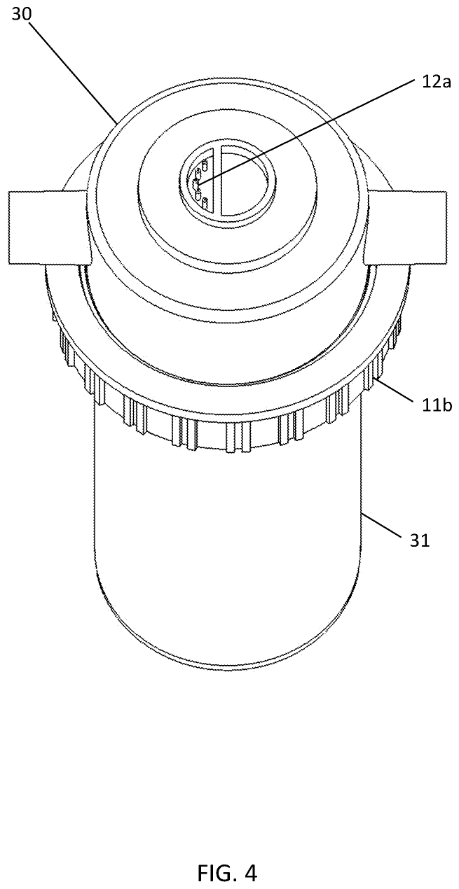

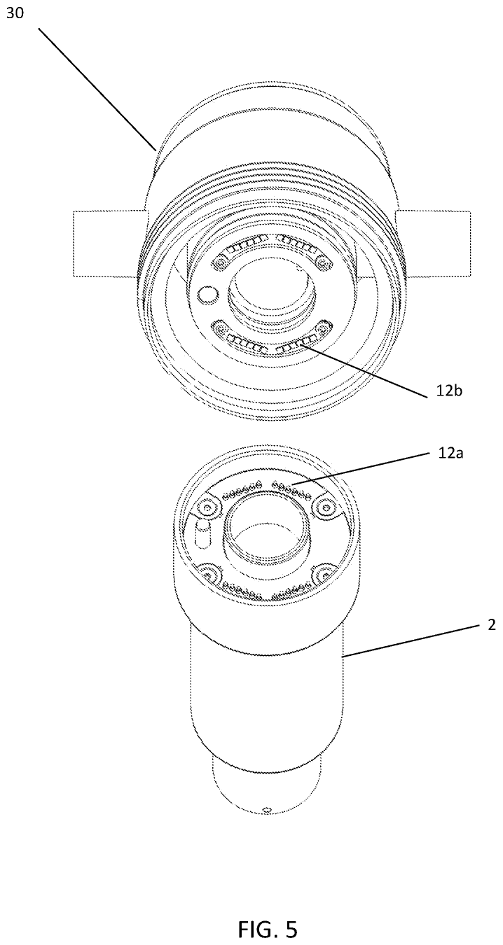

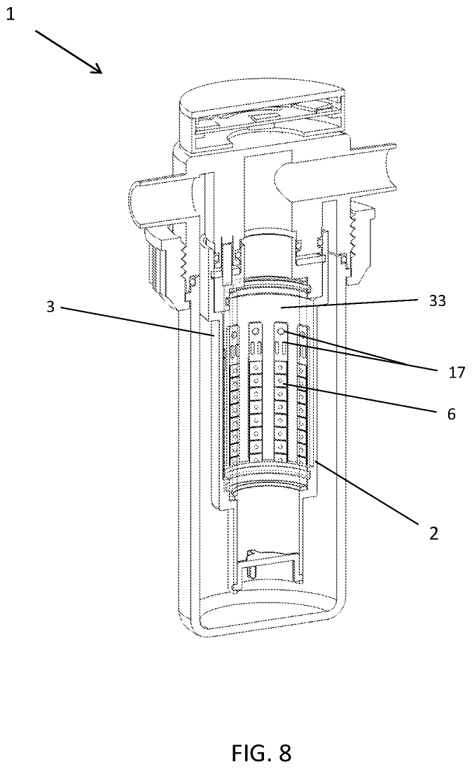

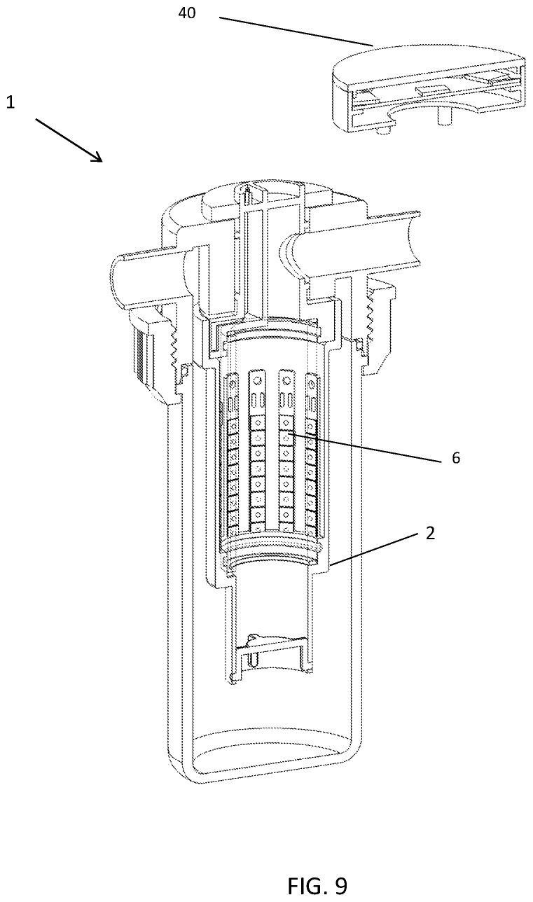

Disclosed embodiments include the detachable light source assembly 2 for treating the fluid with ultraviolet light as part of the fluid treatment system 1, as discussed above, or as a stand-alone device. The light source assembly 2 may include a thermally conductive outer wall 3, an inner wall 4 disposed inside the thermally conductive outer wall 3, an inner space 5 within the inner wall 4 that defines a treatment zone T, an ultraviolet light source 6 (shown in FIGS. 8 and 9) disposed between the thermally conductive outer wall 3 and the inner wall 4 and configured to emit ultraviolet light from behind the inner wall 4 toward the treatment zone T in order to treat the fluid. In embodiments, the ultraviolet light source 6 may be configured to emit the ultraviolet light from behind the inner wall 4 toward a center of the treatment zone T in a radial direction relative to a longitudinal axis A of the light source assembly 2 in order to treat the fluid. The detachable light source assembly 2 may include an electrical contact portion 12a (shown in FIGS. 4 and 5) configured to detachably electrically connect the light source assembly 2 to an electrical power supply of the reactor vessel 25.

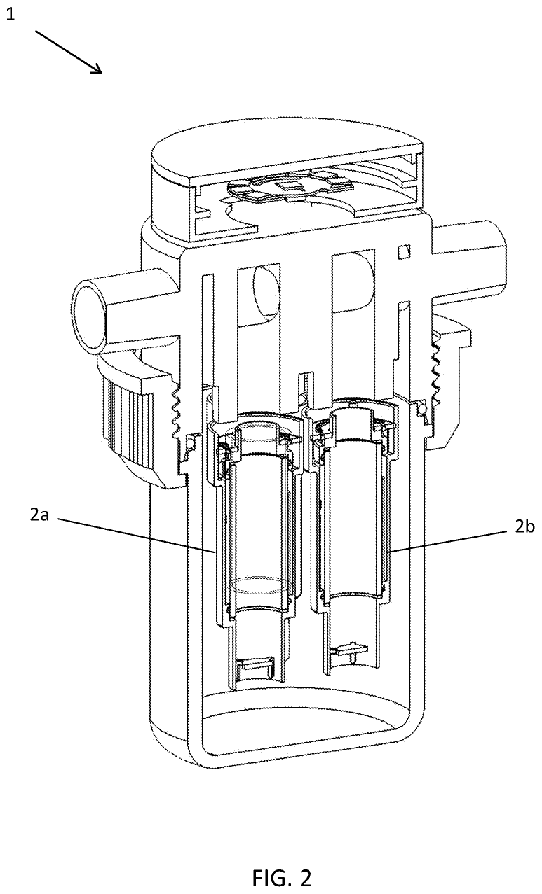

The detachable light source assembly 2 may further include a plurality of light source assemblies. For example, there may be two or three or more light source assemblies such as light source assemblies 2a, 2b, or 2c, as shown in FIGS. 2, 3, and 4. The detachable light source assembly 2 may further include a flow modifying device 10 configured to control a flow of the fluid through the treatment zone T. The flow modifying device 10 may direct fluid to a flow advantageous for treatment by ultraviolet light. The flow modifying device 10 may be, for example, a disk, tube, perforated sheet, and/or a diffuser.

In embodiments, the ultraviolet light source 6 of the detachable light source assembly 2 includes one or more light emitting diodes 12. The light emitting diodes may include ultraviolet LED's, as shown in FIG. 8. The ultraviolet light source 6 may be disposed on an inner surface 33 of the thermally conductive outer wall 3, as best shown in FIG. 8. Disclosed embodiments are not limited to this arrangement. For example, the ultraviolet light source 6, including the one or more light emitting diodes 12, may be disposed on an outer surface of the inner wall 4.

The thermally conductive outer wall 3 may comprise any suitable thermally conductive material that conducts and/or dissipates heat including, but not limited to, a material selected from the group consisting of copper, composite, and stainless steel. The thermally conductive outer wall 3 may be not be homogenous in material. The thermally conductive outer wall 3 may also contain heat-conductive plastic, ceramic, or the like.

The light source assembly 2 may include a reflective material configured to reflect light within the treatment zone. The reflective material may include, but is not limited to, fluoropolymer, TiO₂, SiO₂, and BO₂. The ultraviolet light reflectivity may be in a range of 10% to 100%, 20% to 95%, 30% to 90%, 40% to 80%, or at least 50%.

The inner wall 4 may comprise any suitable ultraviolet transparent material, including, but not limited to, a material selected from the group consisting of polymers such as acrylic and silicone, fluoropolymers, ceramics and glasses such as quartz and fused silica, and specialized ultraviolet glass compositions. In preferred embodiments, the inner wall 4 comprises quartz.

The inner wall 4 may include ultraviolet-transmitting segments. Transmitting segments may be a single area or multiple discrete windows. Transmitting segments may be in longitudinal strips inside the outer diameter of the inner vessel, or may comprise cylindrical elements. For example, ultraviolet LED's may be arranged in strips along the length and around the inner wall 4.

Additionally, the light source assembly 2 may comprise one of more sensors 17, as illustrated in FIG. 8. These sensors may include ultraviolet light intensity sensors such as photodiodes.

### The Reactor Vessel

Disclosed embodiments include a reactor vessel 25 as part of the fluid treatment system 1, as discussed above, or as a stand-alone device. The reactor vessel 25 is configured to receive the at least one detachable light source assembly 2 for treating the fluid with ultraviolet light. The reactor vessel 25 may include an inlet port 26 for receiving a fluid into the reactor vessel 25, and an outlet port 27 for expelling treated fluid from the reactor vessel 25, as shown in FIG. 1A. In alternative embodiments, the reactor vessel 25 may include the reverse configuration, i.e., an inlet port 27a for receiving a fluid into the reactor vessel 25, and an outlet port 26a for expelling treated fluid from the reactor vessel 25, as shown in FIG. 1B.

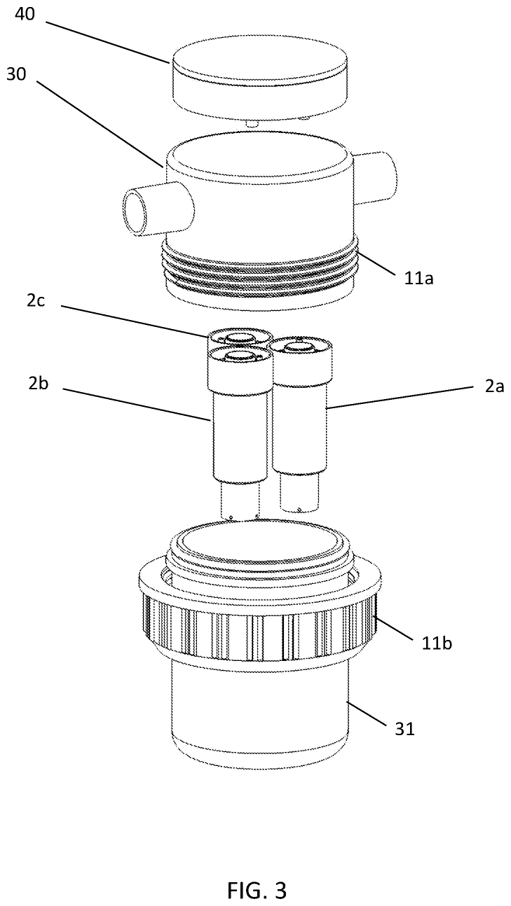

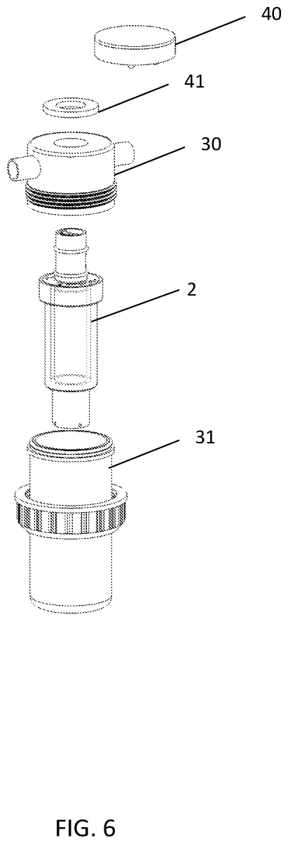

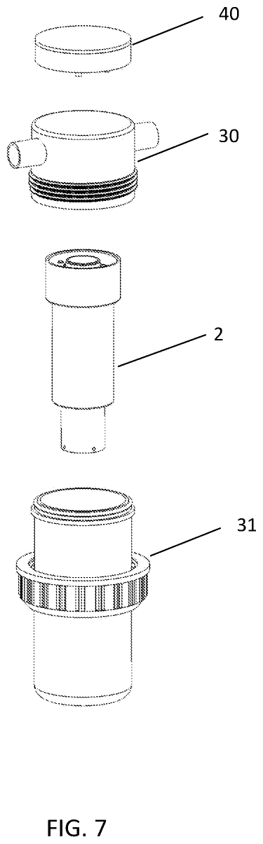

The reactor vessel 25 may include a header portion 30 and a base portion 31, as seen in FIG. 3. The inlet port 26 and/or the outlet 27 port may be a part of a header portion 30. The header portion 30 may include a header connector portion 11a and the base portion 31 may include a base connector portion 11b, the connector portions 11a, 11b configured to connect the header portion 30 and the base portion 31 in a sealed state. The connector portion 11a, 11b may be selected from the group consisting of a press fit, a thread, a bayonet lock, a threaded ring, a sanitary clamp, press fit, or snap fit. In preferred embodiments, the connector portion 11a, 11b is a threaded ring attachment, as illustrated in the top view of the reactor in FIG. 5. The header portion 30 may include a cap 40 and a spacer 41, as shown in FIGS. 6 and 7. The reactor vessel 25 may also include an o-ring 14 to seal the header portion 30 and a base portion 31 with or without the light source assembly 2 disposed therein in the sealed state.

In one embodiment, the fluid may enter the reactor vessel 25 through the inlet port 26 in order to convey water to be treated in the light source assembly 2, as shown by the hollow arrows in FIG. 1A. The influx may be from a holding tank, pipe, body of water, or the like (not shown). The fluid may be pumped, gravity fed, or the like. The reactor vessel 25 may be placed in line with other treatment or filtration devices and components. In this embodiment, the fluid flows to the cooling zone C first and to the treatment zone T. The fluid may flow through the fluid modifier 10, as shown by the hollow arrows. Flow through the fluid modifier 10 may be regulated by the controller 113. In flowing from the cooling zone C to the treatment zone T, the fluid may turn at any suitable angle. For example, the fluid may turn at angle in a range of 90° to 270°, 120° to 240°, 150° to 210°, 170° to 190°, or 180°.

In another embodiment, the fluid may enter the reactor vessel 25 through the inlet port 27a in order to convey water to be treated in the light source assembly 2, as shown by the hollow arrows in FIG. 1B. In this embodiment, the fluid flows to the cooling zone C first and to the treatment zone T. The fluid may flow through the fluid modifier 10, as shown by the hollow arrows. Flow through the fluid modifier 10 may be regulated by the controller 113, as discussed above. In flowing from the treatment zone T to the cooling zone C, the fluid may turn at any suitable angle. For example, the fluid may turn at angle in a range of 90° to 270°, 120° to 240°, 150° to 210°, 170° to 190°, or 180°.

The reactor vessel 25 may include an electrical contact portion 12b configured to electrically connect the reactor vessel 25 to the light source assembly 2, as shown in FIG. 5. A space 28 between a wall of the reactor vessel 29 and the thermally conductive outer wall 3 of the light source assembly 2 defines a cooling zone in which the fluid is cooled by contacting the thermally conductive outer wall 3 before the fluid enters or after the fluid exits a treatment zone of the light source assembly 2 when the light source assembly 2 is in an attached state.

The reactor vessel 25 may have sensors 133 to measure parameters of the fluid send a signal to the controller 113 to determine fluid quality before or after treatment of the reactor, as illustrated in FIG. 1C. The sensors 133 may include one or more temperature sensors disposed at any suitable location throughout the reactor vessel 25, e.g., the inlet port 26, the outlet port 27, and/or in the cooling zone C, in order to monitor fluid temperature, air temperature, LED temperature or other temperature.

### The Fluid Treatment Method

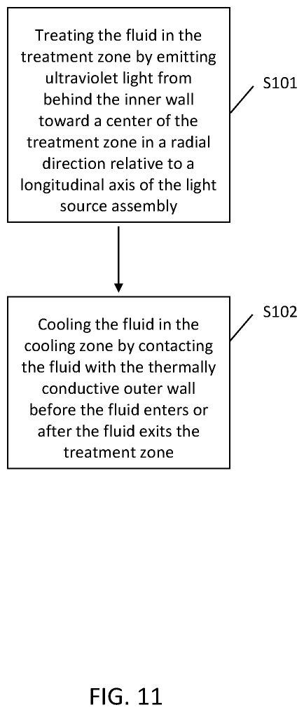

Disclosed embodiments include a method for treating a fluid with ultraviolet light in a fluid treatment system 1, as described above. As shown in FIG. 11, the method includes a step S101 of treating the fluid in the treatment zone by emitting ultraviolet light from behind the inner wall toward a center of the treatment zone in a radial direction relative to a longitudinal axis of the light source assembly. The method further includes a step S102 of cooling the fluid in the cooling zone by contacting the fluid with the thermally conductive outer wall before the fluid enters or after the fluid exits the treatment zone.

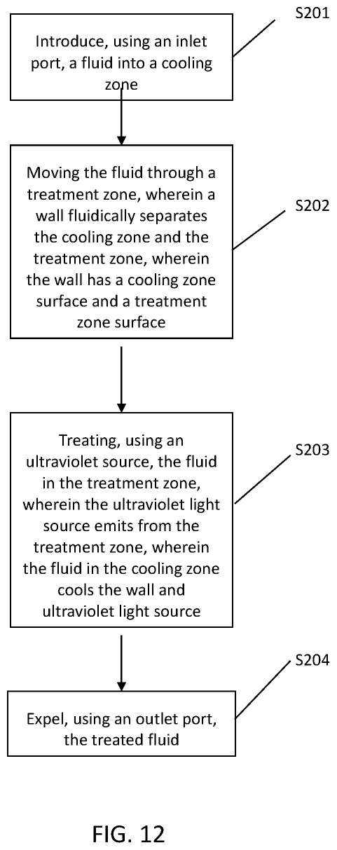

Referring now to FIG. 12, disclose methods may include treating a fluid using ultraviolet light. The fluid may enter through the inlet port 26 (S201). From the inlet port 27, the fluid may enter the cooling zone (S202). From the cooling zone, the fluid may enter a treatment zone (S203). The fluid modifier 10 may be present between the cooling and treatment zones. The fluid may undergo ultraviolet illumination or treatment in the treatment zone. From the treatment zone, the fluid may exit the reactor from an outlet port (S204).

### The Communication Network

The fluid treatment system 1 may connect to a communication network 100 including a controller 113 for executing control functions of the system, as shown in FIG. 10. The controller 113 may be configured to control and/or adjust operation of the fluid treatment system 1, light source assembly 2, and/or reactor vessel 25. The controller 113 may also be configured to receive sensor input and calculate and/or determine values of relevant input/output parameters.

For example, sensor data associated with one of a plurality of light source, temperature, or the like may be received by the controller 113. Sensor data may include transmittance values, power outputs, electrical characteristics (e.g., voltage, current, etc.), environmental characteristics (e.g., temperature, turbidity of the fluid, etc.), flow rate, ultraviolet intensity, time, voltage, and optical absorbance or any other characteristics of the light source or surrounding environment that may identify a change in system performance, fluid properties, or output by a light source. For example, the ultraviolet light source assembly may include a light sensor that can measure a value of irradiance produced by the light source. The light sensors may be of different wavelengths than the wavelength of the treatment light source. For example, the light sensors may be of any wavelength from visible to infrared sensors, whereas the wavelength of the treatment light source is within the ultraviolet wavelength. Additionally, the light sensor wavelengths may be ultraviolet sensors. The ultraviolet light source assembly may include more than one light sensor, with each light sensor measuring an output of the light source at a different location, for example, before or after passing through the fluid.

Additionally, in an embodiment, the sensor data provided by an ultraviolet light source may include location data that identifies the location of the light source in an array or a grouping of reactors. Identification of the location of the light source may assist in troubleshooting, maintenance, or the like. Neighboring reactors may be adjusted to account for a reactor or reactor portion that is underperforming. For example, if an underperforming single LED or LED assembly is detected, then treatment may be compensated by increasing current or power to the underperforming unit or to another unit. Similarly, if an underperforming single LED or LED assembly is detected, an alarm or notification may be provided.

The controller 113 may have default values for any of the characteristics that can be measured or identified from any of the sensors provided within the fluid treatment system 1, light source assembly 2, and/or reactor vessel 25. When the controller 113 receives information from the sensor, it may compare that information to the default values. If the difference between the received information and the default values is above a predetermined threshold, the controller 113 may identify that as a change in output. As another example, the controller 113 may compare sensor information between different sensors in the fluid treatment system 1, light source assembly 2, and/or reactor vessel 25. Differences in sensor information among a group of sensors may indicate a change in output of one or more light source(s). For example, the controller 113 may determine that differences in sensor information between neighboring or adjacent light sources above a predetermined threshold may indicate a change in one or more of the light source(s). Thus, a light source that is underperforming or providing a lowered output may result in sensor data identifying as much. Similarly, a light source that is providing an output greater than a previous output may result in sensor data identifying as much.

For example, a sensor may collect information on the numbers of hours a light source or reactor has been turned on. The controller 113 may have a predetermined threshold for number of hours of service life a light source may have. If a light source is nearing the end of a service life, then the controller 113 may determine the light source is nearing an hour limit, and send a command to the processor or power supply to turn off, reduce power, or the like, to the light source nearing the hour limit. Additionally or alternatively, the controller 113 may send a command to a processor or power supply of a light source that has a decrease in ultraviolet light output commanding it to increase power to the light source or another light source. Hour usage and intensity are illustrative examples, and other sensors with other associated light source or fluid characteristics are disclosed.

In an embodiment, the modulation of a power supply may bring a light source back to a desired functioning parameter. The light source is able to provide instructions to the power supply so that the light source can achieve maximum useful lifespan. Modulation of the power supply may include modulating power output, modulating current output, modulating voltage output, modulating power supply cycles or cycle times, output pulse width or duty cycle, or modulating any other characteristic of the power supply.

Network 100 typically includes one or more of a WWAN transceiver 115 and a WLAN transceiver 116 for connecting to various networks, such as telecommunications networks and wireless Internet devices, e.g., access points. Additionally, devices 112 are commonly included, e.g., a transmit and receive antenna, oscillators, PLLs, etc. Network 100 includes input/output devices 117 for data input and display/rendering (e.g., a computing location located away from the single beam system that is easily accessible by a user). Network 100 also typically includes various memory devices, for example flash memory 118 and SDRAM 119.

The controller 113 may alert a user via a network 100. This alert may occur whether a power supply modulation occurs or not. An alert may be in a form of audio, visual, data, storing the data to a memory device, sending the output through a connected or wireless system, printing the output or the like. The controller 113 may log information such as a sensor output value, location, a corrective action, time, date, number of cycles, or the like. The alert or log may be automated, meaning the system may automatically output whether a correction was required or not. The controller 113 may also have associated alarms, limits, or predetermined thresholds. Alarms or logs may be analyzed in real-time, stored for later use, or any combination thereof.

While various other circuits, circuitry or components may be utilized in information handling devices, with regard to fluid treatment according to any one of the various embodiments described herein, an example is illustrated in FIG. 10. The network 100 may include a measurement system on a chip design found, for example, a particular computing platform (e.g., mobile computing, desktop computing, etc.). Software and processor(s) are combined in a single chip. Processors comprise internal arithmetic units, registers, cache memory, busses, I/O ports, etc., as is well known in the art. Internal busses and the like depend on different vendors, but essentially all the peripheral devices 112 may attach to a single chip. The network 100 combines the controller 113, memory control, and I/O controller hub all into a single system. Also, common interfaces, for example, include SDIO and I2C.

The network 100 may include a battery management unit, BMU, which manage power as supplied, for example, via a rechargeable battery 114, which may be recharged by a connection to a power source (not shown). In at least one design, a single chip, is used to supply BIOS like functionality and DRAM memory. The network 100 may also include a power management circuit 105.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a serial connection such as USB or RS485 connections.

Example embodiments are described herein with reference to the figures, which illustrate example methods, devices and products according to various example embodiments. It will be understood that the actions and functionality may be implemented at least in part by program instructions. These program instructions may be provided to a processor of a device or other programmable data processing device to produce a machine, such that the instructions, which execute via a processor of the device, implement the functions/acts specified.

It should be understood that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A fluid treatment system for treating a fluid with ultraviolet light, the system comprising:
at least one detachable light source assembly having a thermally conductive outer wall, an inner wall disposed inside the thermally conductive outer wall and surrounding an inner space that defines a treatment zone, and an ultraviolet light source disposed between the thermally conductive outer wall and the inner wall and configured to emit ultraviolet light toward a center of the treatment zone in order to treat the fluid; and
a reactor vessel having an inlet port for receiving a fluid into the reactor vessel and an outlet port for expelling treated fluid from the reactor vessel, the reactor vessel configured to receive the light source assembly in an attached state such that a space between a wall of the reactor vessel and the thermally conductive outer wall of the light source assembly defines a cooling zone in which the fluid contacts the thermally conductive outer wall before the fluid enters or after the fluid exits the treatment zone to thereby cool the thermally conductive outer wall.

2. The fluid treatment system according to claim 1, further comprising a plurality of light source assemblies,
wherein the reactor vessel is configured to receive the plurality of light source assemblies in an attached state.

3. The fluid treatment system according to claim 1, further comprising a flow modifying device disposed on a fluid entry side of the light source assembly, the flow modifying device configured to control a flow of the fluid through the treatment zone.

4. The fluid treatment system according to claim 1, wherein the reactor vessel includes a header portion and a base portion, the header portion and the base portion each further includes a connector portion configured to connect the header portion and the base portion in the attached state.

5. The fluid treatment system according to claim 4, wherein the connector portion is selected from the group consisting of a press fit, a thread, a bayonet lock, a threaded ring, and a sanitary clamp.

6. The fluid treatment system according to claim 4, further comprising an o-ring to seal the header portion and the base portion and in the attached state.

7. The fluid treatment system according to claim 1, wherein the reactor vessel and the light source assembly each further includes an electrical contact portion configured to electrically connect the light source assembly and the reactor vessel.

8. The fluid treatment system according to claim 1, wherein the ultraviolet light source comprises a plurality of light emitting diodes.

9. The fluid treatment system according to claim 1, further comprising a controller configured to adjust an intensity of the ultraviolet light source based on at least one parameter selected from the group consisting of time, intensity, flow rate, voltage, temperature, ultraviolet intensity, and optical absorbance.

10. The fluid treatment system according to claim 1, wherein the fluid turns 180 degrees flowing from the cooling zone to the treatment zone.

11. The fluid treatment system according to claim 1, wherein light source assembly has a cylindrical shape or a rectangular cross-sectional shape.

12. The fluid treatment system according to claim 1, wherein the thermally conductive outer wall comprises a material selected from the group consisting of copper, composite, and stainless steel.

13. The fluid treatment system according to claim 1, wherein an inner surface of the light source assembly includes a reflective material configured to reflect light within the treatment zone.

14. The fluid treatment system according to claim 13, wherein the reflective material is at least one selected from the group consisting of fluoropolymer, TiO₂, SiO₂, and BO₂.

15. The fluid treatment system according to claim 1, wherein the ultraviolet light source is disposed on an inner surface of the thermally conductive outer wall and is configured to emit the ultraviolet light toward the center of the treatment zone in a radial direction relative to a longitudinal axis of the light source assembly.

16. The fluid treatment system according to claim 1, wherein the inner wall comprises a quartz material.

17. The fluid treatment system according to claim 1, wherein the fluid is water in a water treatment facility.

18. A method for treating a fluid with ultraviolet light in a fluid treatment system, the system comprising (i) at least one detachable light source assembly having a thermally conductive outer wall, an inner wall disposed inside the thermally conductive outer wall and surrounding an inner space that defines a treatment zone, and an ultraviolet light source disposed between the thermally conductive outer wall and the inner wall, and (ii) a reactor vessel having an inlet port for receiving a fluid into the reactor vessel and an outlet port for expelling treated fluid from the reactor vessel, the reactor vessel configured to receive the light source assembly in an attached state such that a space between a wall of the reactor vessel and the thermally conductive outer wall of the light source assembly defines a cooling zone, the method comprising:
treating the fluid in the treatment zone by emitting ultraviolet light toward the treatment zone; and
cooling the fluid in the cooling zone by contacting the fluid with the thermally conductive outer wall before the fluid enters or after the fluid exits the treatment zone to thereby cool the thermally conductive outer wall.

19. A light source assembly for treating a fluid with ultraviolet light, the assembly comprising:
a thermally conductive outer wall;
an inner wall disposed inside the thermally conductive outer wall;
an inner space surrounded by the inner wall that defines a treatment zone;
an ultraviolet light source disposed between the thermally conductive outer wall and the inner wall and configured to emit ultraviolet light toward the treatment zone in order to treat the fluid; and
an electrical contact portion configured to detachably electrically connect the light source assembly to an electrical power supply.

20. The light source assembly according to claim 19, wherein the inner wall comprises an ultraviolet-transmissive material selected from the group consisting of quartz, sapphire, and fluoropolymer.

21. The light source assembly according to claim 19, further comprising a sensor configured measure an intensity of the ultraviolet light.

22. The light source assembly according to claim 19, wherein the inner wall includes at least one portion with ultraviolet light reflectivity of at least 50%.

23. The light source assembly according to claim 19, further comprising a flow modifying device disposed on a fluid entry side of the light source assembly, the flow modifying device configured to control a flow of the fluid through the treatment zone.

24. A reactor vessel configured to receive at least one detachable light source assembly for treating a fluid with ultraviolet light, the reactor vessel comprising:
an inlet port for receiving a fluid into the reactor vessel;
an outlet port for expelling treated fluid from the reactor vessel;
a header portion and a base portion, the header portion and the base portion each further including a connector portion configured to connect the header portion and the base portion in an attached state; and
an electrical contact portion configured to electrically connect the reactor vessel to the light source assembly,
wherein a space between a wall of the reactor vessel and a thermally conductive outer wall of the light source assembly defines a cooling zone in which the thermally conductive outer wall is cooled by contacting the fluid with the thermally conductive outer wall before the fluid enters or after the fluid exits a treatment zone of the light source assembly when the light source assembly is in an attached state.
